(19) United States
(12) Patent Application Publication
(10) Pub. No.: US 2014/0328898 A1
(43) Pub. Date: Nov. 6, 2014

(54) Nano-Liposomal Formulations and Methods of Use

(71) Applicants: CHILDREN'S NATIONAL MEDICAL CENTER, Washington, DC (US); UNIVERSITY OF MARYLAND, COLLEGE PARK, College Park, MD (US)

(72) Inventors: Renee Hood, Baltimore, MD (US); Eric Kendall, Washington, DC (US); Donald Lad DeVoe, Bethesda, MD (US); Julia Cole Finkel, Washington, DC (US); Zenaide Quezado, Washington, DC (US); Mariana Mafra Junqueira, Washington, DC (US)

(73) Assignees: CHILDREN'S NATIONAL MEDICAL CENTER, Washington, DC (US); UNIVERSITY OF MARYLAND, COLLEGE PARK, College Park, MD (US)

(21) Appl. No.: 14/212,874

(22) Filed: Mar. 14, 2014

Related U.S. Application Data
(60) Provisional application No. 61/788,249, filed on Mar. 15, 2013.
(60) Provisional application No. 61/788,224, filed on Mar. 15, 2013.

(51) Int. Cl. A61K 9/127

(52) U.S. Cl. CPC A61K 9/127
USPC 424/450

(57) ABSTRACT

A method for transdermal drug delivery provides for the topical administration of liposome encapsulated nanoparticles. The encapsulated nanoparticles define a nearly monodisperse population of liposomes having an average diameter within a selected size range. Liposomal nanoparticle formulations and methods of treatment therewith are also provided.

### CROSS-REFERENCE TO RELATED APPLICATIONS

This application is based on U.S. Provisional Patent Application Ser. No. 61/788,224, filed Mar. 15, 2013, and U.S. Provisional Patent Application Ser. No. 61/788,249, filed Mar. 15, 2013, which applications are both incorporated herein by reference in their entireties and to which priority is claimed.

### FIELD OF THE INVENTION

The present invention relates to methods of transdermal drug delivery by the topical administration of liposome encapsulated nanoparticles. The encapsulated nanoparticles are synthesized and selected utilizing techniques that generate small, nearly monodisperse populations of liposomes having an average diameter within a target size range capable of passive transdermal diffusion. Liposomal nanoparticle formulations and methods of treatment therewith are also provided.

### BACKGROUND OF THE INVENTION

Many medical procedures require local anesthesia, analgesia and/or other therapeutic drugs which are administered orally or via hypodermic injection. Hypodermic delivery via a needle permits relatively fast and deep penetration of the drug through the dermal layers. However, such administration may be painful and difficult for some patients, particularly infants and children. Moreover, administration via needles and/or catheters may increase the possibility of infection and septicemia. As such, relatively painless and easy administration of such drugs would be highly desirable, particularly for pediatric patients.

Transdermal delivery of therapeutics is poised to have a substantial impact on drug delivery, and would be an appealing alternative to hypodermic injection, with clinical applications spanning from pain management to dementia (Prausnitz et al. (2008) “Transdermal drug delivery,” Nature biotechnology 36(II):1261-8). Various techniques to deliver macromolecules through the skin have been attempted, including chemical enhancers, electroporation, cavitational ultrasound microneedles, thermal ablation and microdermabrasion (Arora et al. (2008) “Micro-scale devices for transdermal drug delivery,” International J. Pharmaceutics 364(2):227-36). Such techniques can temporarily increase porosity of the stratum corneum (SC), the 10-20 μm thick protective layer of the epidermis, in an attempt to enhance drug transport. However, such methods require active disruption of the skin and do not allow controlled doses to be delivered over long time periods. Similarly, non-invasive active methods, such as ionophoresis and ultrasound, require specialized equipment and only enhance porosity for a relatively short period. As such, although some improvements have been observed using such techniques, each involves disrupting the SC in order to reach the deeper tissues and/or fails to achieve acceptable delivery results.

Thus, attempts to provide transdermal drug delivery have failed to achieve acceptable results. In particular, effective delivery of drugs through the skin has been hampered by poor diffusive transport across the SC (Schreier et al. (1994) “Liposomes and niosomes as topical drug carriers: dermal and transdermal drug delivery,” J. Control Release 30:1-15; Cevc G. (2004) “Lipid vesicles and other colloids as drug carriers on the skin,” Adv. Drug Deliv. Rev. 56:675-711). Indeed, even when employing chemical penetration enhancers, a broad class of skin disrupting molecules including a variety of surfactants, transdermal drug delivery has not been successful (Prausnitz et al. (2008), supra, Nature biotechnology 36(141261-8; Karande et al. (2005) “Design principles of chemical penetration enhancers for transdermal drug delivery,” PNAS 102:4688-4693).

Thus, conventional attempts to provide transdermal drug delivery have failed to demonstrate sufficiently fast and deep penetration through the skin layers. Moreover, such attempts have raised safety issues, particularly the biodegradability of inorganic nanomaterials which can be highly toxic once taken up and retained by the reticuloendothelial system. As such, conventional techniques have failed to eliminate the need for delivery via needle injection.

### SUMMARY OF THE INVENTION

The present invention relates to methods of transdermal drug delivery, and pharmaceutical compositions for transdermal drug delivery, wherein liposome drug formulations comprising nanoparticle populations of selected and tunable size are provided. The liposome nanoparticles have the ability to passively cross through the SC for transdermal drug delivery. Topical application of therapeutics provides an avenue for painless, noninvasive delivery of molecules for various clinical conditions. Unlike traditional methods, which utilize polydisperse liposomes too large to traverse dermal layers (typically >80 nm), the disclosed methods of the present invention utilize populations of small, nearly-monodisperse liposomes having diameters within a target size range, preferably less than 50 nm, more preferably less than about 40 nm, which are capable of passive transdermal drug delivery.

The present invention also relates to anesthetics, analgesics and/or other compositions encapsulated in liposome vesicles or envelopes, and which are passively deliverable to a patient via the cutaneous route. Thus, the disclosed compositions may be applied directly to the skin surface, thereby providing topical application for local anesthesia, analgesia and/or other therapeutics. The encapsulated drugs provide topical local analgesia and additionally central analgesia upon reaching the bloodstream. The encapsulated drug formulations are able to quickly penetrate the skin layers, promoting fast drug delivery (e.g., fast onset anesthesia) while also providing long duration via slow drug release and thus an improved safety profile.

The present invention also relates to methods of treatment utilizing the liposome encapsulated compositions. The liposome encapsulated drug formulations may be applied topically, systemically, or for anesthesia blocks. Drug delivery via the cutaneous route is particularly advantageous for some applications, improving topical anesthesia for minor procedures (needle puncture, biopsies, etc.), and providing better procedure-related pain relief in patients, particularly children, as compared to conventional methodologies. Further, a number of preparations may be used to provide topical anesthesia prior to needle sticks. The cutaneous route may also be utilized to deliver drugs systemically. Liposomal size and the specific drug formulations may be optimized (e.g., such as utilizing microfluidic techniques) for a particular site of administration and for desired drug delivery characteristics.

A method of transdermal drug delivery according to an embodiment of the present invention comprises the steps of: encapsulating an agent in liposome vesicles to form a population of encapsulated nanoparticles having an average diameter within a target size range; and topically administering the encapsulated nanoparticles for transdermal delivery of the agent to a patient.

According to embodiments of the present invention, the average diameter of the liposome encapsulated nanoparticles is selected during encapsulation. The average diameter may be selected based on a target drug delivery and release profile. According to some embodiments, the population of encapsulated nanoparticles has a polydispersity in nanoparticle diameter of less than about 20%, more preferably less than about 10%, more preferably less than about 5%, more preferably less than about 2%. In some implementations, the average nanoparticle diameter is less than about 50 nm, more preferably less than about 40 nm, and more preferably having a diameter of between about 20 nm and about 40 nm.

In some implementations, the agent is selected from the group consisting of an anesthetic, an analgesic, an antibiotic, a hormone, or some other therapeutic or diagnostic agent. In some implementations, the agent provides local analgesia in an area of topical application. In other implementations, the agent is transdermally delivered to the bloodstream of the patient and provides central analgesia.

A method of transdermal drug delivery according to another embodiment comprises the steps of: encapsulating a first agent in liposome vesicles to form a first population of encapsulated nanoparticles having a first average diameter; encapsulating a second agent in liposome vesicles to form a second population of encapsulated nanoparticles having a second average diameter different from the first average diameter; and topically administering the first and second populations of encapsulated nanoparticles for transdermal delivery of the first and second agents to a patient.

According to some embodiments, the first agent is an anesthetic, an analgesic, an antibiotic, or a hormone. The second agent may also be an anesthetic, an analgesic, an antibiotic, or a hormone, and either the same or different from first agent.

A pharmaceutical composition according to the present invention comprises a population of liposome encapsulated nanoparticles having an average diameter within a target size range capable of passive transport across a stratum corneum of a subject for transdermal delivery of the nanoparticles, and a pharmaceutically acceptable carrier. In some implementations, the encapsulated nanoparticles include an anesthetic, an analgesic, an antibiotic, a hormone, or some other therapeutic or diagnostic agent.

According to embodiments of the present invention, the average diameter of the liposome encapsulated nanoparticles of the composition have a polydispersity in nanoparticle diameter of less than about 20%, more preferably less than about 10%, more preferably less than about 5%, more preferably less than about 2%. In some implementations, the average nanoparticle diameter is less than about 50 nm, more preferably less than about 40 nm, and more preferably having a diameter of between about 20 nm and about 40 nm.

According to some embodiments, the composition includes first and second populations of encapsulated nanoparticles, wherein the first population includes nanoparticles having a first average diameter within a first target size range, and the second population includes nanoparticles having a second average diameter within a second target size range. The second population of encapsulated nanoparticles may include a locally effective anesthetic, analgesic, antibiotic, hormone, or other agent.

In some implementations, the second target size range is capable of passive transport across the stratum corneum of the subject for transdermal delivery of the nanoparticles. In some implementations, the first population of encapsulated nanoparticles has a first drug release profile, and the second population of encapsulated nanoparticles has a second drug release profile different than the first drug release profile.

### BRIEF DESCRIPTION OF THE DRAWINGS

The patent or application Mile contains at least one drawing/photograph executed in color. Copies of this patent or patent application publication with color drawing(s) will be provided by the Office upon request and payment of the necessary fee,

FIG. 1 shows graphically a comparison of liposomes made via one-step microfluidic focusing at different flow rate ratios (FRRs) and traditional bulk homogenization following multiple filtration steps. The microfluidic method enables production of lipid vesicles of tunable size, which exhibit substantially narrower size distributions as well as lower size limitations as compared to bulk methods.

FIG. 2 shows schematically an exemplary microfluidic process of liposome production for producing liposome nanoparticles suitable for use with the methods and compositions of the present invention. FIG. 2(a) illustrates an exemplary liposome synthesis chip made of a PDMS mold from an epoxy-based negative photoresist (SU-8 master). FIG. 2(b) illustrates a numerical simulation of hydrodynamic focusing in the device in which a center stream of ethanol and solvated lipid is sheathed by two oblique streams of aqueous buffer. As lipids slowly diffuse from their solvated state in ethanol into the aqueous buffer, they systematically self-assemble into vesicles.

FIG. 3 shows graphically volume-weighted distributions of populations of liposomes produced using microfluidic techniques. Liposomes with diameters both above and below (FRR 5 and FRR 50, respectively) the size range expected to passively traverse the dermal layer were provided.

FIG. 4 are bright field/fluorescence overlay images (plates A and B) of porcine tissue, and single channel fluorescence images (plates C and D) of porcine tissue exposed to liposomes encapsulating lipophilic cationic indocarbocynanine dye (DiI). In the case of large liposomes (287.1 nm), the lipophilic dye did not penetrate deep into the tissue (plates A and C), while smaller liposomes (32.4 nm) transported intact across the SC (plates B and D).

FIG. 5 show graphically representative sections of the porcine tissue of FIG. 4 analyzed for dye penetration depth, showing that large (FIG. 5A) and small (FIG. 5B) liposomes revealed a quantitative difference in depth penetration of the lipophilic dye across multiple tissue samples.

FIG. 6 shows graphically volume-weighted size distributions of microfluidic-enabled (FIG. 6(a)) PEGylated and (FIG. 6(b)) anionic liposomes, revealing narrow size distributions over the full size range from 31 nm to 308 nm.

FIG. 7 are brightfield/fluorescence image overlays (top plates A, B, C, D, E, F) and single-channel fluorescence images (bottom plates G, H, I, J, K, L) for microtomed tissue sections following 15 minute application of PEGylated or anionic liposome samples of varying diameters containing DiI lipophilic dye. Significant dye penetration past the SC was observed with the smallest liposomes (31 nm diameter PEGylated and 41 nm anionic liposomes), while dye from the larger vesicles did not appear to cross the SC, indicating size-based passive transport independent of surface charge.

FIG. 8 shows graphically DiI fluorescence intensity plot profiles for (FIG. 8(a)) PEGylated liposomes and (FIG. 8(b)) anionic liposomes as a function of porcine skin tissue penetration depth. Measurements were performed 15 minute following liposome application. Each curve is representative of an average of five (5) regions of interest (ROIs) per image.

FIG. 9 shows graphically percentage of total DiI fluorescence signal seen below the SC for the different sizes of PEGylated and anionic liposomes. Each plot reflects the average profile extracted from five (5) ROIs per tissue section, with error bars reflecting standard deviation. SC thickness, estimated from averaged manual measurements using brightfield images of each tissue, ranged from 15 μm to 40 nm, in general agreement with previously reported values for porcine skin. The small 31 nm PEGylated liposomes pass the SC in large numbers (91%), which is up to 590% greater than the larger 105 nm to 308 nm diameter liposomes. The small 41 nm anionic liposomes also reveal 65% of their total DiI signal under the SC, which is 200% greater than observed with 256 nm diameter liposomes of the same composition.

FIG. 10 are brightfield images of three (3) representative tissue regions following application of 31 nm PEGyated liposomes to porcine skin tissue (top plates A, B, C), together with matched single channel fluorescence images for lipophilic DiI (middle plates D, E, F, showing penetration of a first/red dye) and hydrophilic SF (bottom plates G, H, I, showing penetration of a second/green dye). Similar fluorescence distributions for both dyes are seen across multiple tissue sections, indicating successful penetration of intact liposomes through the epithelium.

FIG. 11 illustrates graphically penetration depth profiles of lipophilic and hydrophilic liposomal dyes within a tissue section following 15 minutes application of 31 nm PEGyated liposomes simultaneously loaded with both dyes. Each curve is representative of an average of five (5) ROIs per image. A Pearson's correlation coefficient of p=0.92 reveals a high degree of colocalization between the dyes.

### DETAILED DESCRIPTION OF THE INVENTION

The present invention is directed to a method of delivering molecules to deeper tissues of the skin by passively traversing the SC using nanoparticles having a selected size. Topical application of nanoparticles for passive transdermal delivery of active reagents through the skin for effective treatment of a variety of clinical conditions is achieved, offering increased drug loading, sustained release, and the potential for tissue-specific targeting. The disclosed nanoparticles and drug formulations demonstrate enhanced drug penetration and substantially expand the range of molecules that may be passively delivered to a patient via the transdermal route in a noninvasive manner.

The structure of the SC includes lamellar lipid regions that present sub-nanometer intercellular spaces which can be widened in the presence of nanoparticle colloids to pores with dimensions on the order of several tens of nanometers (Cevc G (2004), supra, Adv. Drug Deliv. Rev. 56:675-711; Bouwstra et al. (2003), supra, Prog. Lipid Res. 42:1-36). Liposomes, nanoscale vesicles composed of natural physiological and completely biodegradable lipids, exhibit low toxicity and excellent tolerability, as well as enhanced permeation and chemical stability. As such, nanoscale liposomes with lipid bilayers encapsulating aqueous internal volumes offer high loading of both hydrophilic and amphipathic drugs, low toxicity, and tunable stability.

A population of nearly monodisperse liposomes having sizes within a specific target range are utilized for passive transdermal delivery of an agent (e.g., a therapeutic agent, reagent, or drug formulation). In a preferred embodiment, the liposomes are synthesized in accordance with microfluidic techniques. The resulting liposome formulations have the ability to transport through the SC, the uppermost layer of skin consisting primarily of a lipid/protein matrix which is the principal barrier to drug penetration through the skin (see Cevc G. (2004), supra, Adv. Drug Deliv. Rev. 56:675-711). Liposomal encapsulation of lipophilic and hydrophilic drugs improves drug safety profile and allows for a relatively prolonged release.

Many novel formulations may be provided which are designed for a specific administration route and/or with a specific treatment effect and goal. For example, liposome encapsulated drugs are suitable for a variety of applications, such as for anti-fungal medications, chemotherapeutic drugs, topical anesthetics and analgesia, sedation, and nerve blocks. Previous attempts to provide transdermal delivery systems failed to pass through the SC in therapeutically effective amounts and therefore have not yielded the expected benefits. Indeed, a number of studies exploring the dermal transport of conventional, larger sized liposomes (from about 60 nm to several micrometers in diameter) revealed poor transport through the SC (Du Plessis et al. (1994), “The influence of particle size of liposomes on the deposition of drug into skin,” Int. J. Pharm. 103:277-282; Prow et al. (2011) “Nanoparticles and microparticles for skin drug delivery,” Adv. Drug Deliv. Rev. 63:470-491; Sudhakar et al. (2012) “Ethosomes as Non-Invasive Loom for Transdermal Drug Delivery Systems,” Sebastian M, Ninan N, Haghi A K, editors, Nanomedicine and Drug Delivery, Apple Academic Press, pp. 1-15; Sentjurc et al. (1999) “Liposomes as a topical delivery system: the role of size on transport studied by the EPR imaging method,” J. Control Release 59:87-97). Such previous studies failed to demonstrate that lipid vesicles are able to traverse the SC in significant numbers; nor do such previous studies provide any evidence of intact liposome passage through the SC.

As a result, the application of lipid nanoparticles for transdermal drug delivery has previously focused on flexible liposomes such as transfersomes (Cevc et al. (1998) “Ultraflexible vesicles, Transfersomes, have an extremely low pore penetration resistance and transport therapeutic amounts of insulin across the intact mammalian skin,” Biochim. Biophys. Acta. 1368:201-215) and ethosomes (Touitou et al. (2000) “Ethosomes—novel vesicular carriers for enhanced delivery: characterization and skin penetration properties,” J. Control. Release 65:403-418), which incorporate surfactants or alcohols to impart a high degree of flexibility to the relatively large vesicles. However, for systemic delivery through the bloodstream, such nanoparticles did not prove effective since large and flexible liposomes are subject to rapid opsonization and phagocytotic clearance. Furthermore, whereas both pharmacokinetics and biodistribution of traditional liposomes have been studied and optimized, the behaviors of flexible liposomes remain largely unknown. Moreover, recent evidence indicates that ultraflexible transfersomes are highly compromised by passage through the skin and thus not suitable for transdermal delivery of intact vesicles (Brewer et al. (2013) “Spatially resolved two-color diffusion measurements in human skin applied to transdermal liposome penetration,” J. Invest. Dermatol. 133:1260-1268).

In contrast, the disclosed formulations of the present invention, including local anesthetics, analgesics, antibodies, and other therapeutics encapsulated inside of liposome vesicles or envelopes having selected size characteristics, are able to effectively pass through the SC barrier. Thus, transdermal drug delivery by topical administration of selected drugs (e.g., such as for local anesthesia and pain relief) is achieved. In addition, the hydrophobic characteristic of the liposome encapsulated drugs of the present invention allow them to cross the skin layers substantially faster and more effectively as compared to the drug itself. The liposome encapsulated drugs provide for relatively slow drug release, thus avoiding high concentrations in the blood stream after administration. Unlike typical inorganic nanoparticles, liposomes are non-toxic, and able to encapsulate both hydrophilic and lipophilic compounds, and thus are a highly attractive nanocarrier for dermal transport of drugs.

According to some embodiments of the present invention, the liposome encapsulated nanoparticles have a diameter of less than about 50 nm, more preferably within the range of about 20 nm to about 40 nm. Such smaller liposome nanoparticles successfully penetrate the primary dermal barrier. With the benefits of liposomal drug delivery, together with the ability to generate small, narrowly distributed populations of liposomes within the size range of interest, effective transdermal drug delivery is achieved.

In contrast, conventional methods have utilized liposomes populations (e.g., produced via alcohol injection, membrane extrusion, detergent dialysis, and sonication) that are both too large (e.g., greater than 80 nm), and too polydisperse with typical populations exhibiting high variance in size and with distributions skewed toward the larger diameter molecules (e.g., greater than 80 nm). As such, such liposome populations have exhibited poor performance for dermal drug delivery.

According to a preferred embodiment, liposome synthesis and encapsulation of local anesthetics, analgesics and other formulations are provided utilizing a microfluidic synthesis technique. The microfluidic synthesis technique offers the ability to generate relatively small liposome vesicles having the desired average diameter. Preferably, the average liposome diameter within a population is less than 60 nm, more preferably less than 50 nm, and more preferably less than 40 nm. In some applications, the average liposome diameter within a population generated in accordance with the disclosed microfluidic techniques is between about 20 nm and about 50 nm, more preferably between about 20 nm and about 40 nm, and more preferably between about 30 nm and about 40 nm.

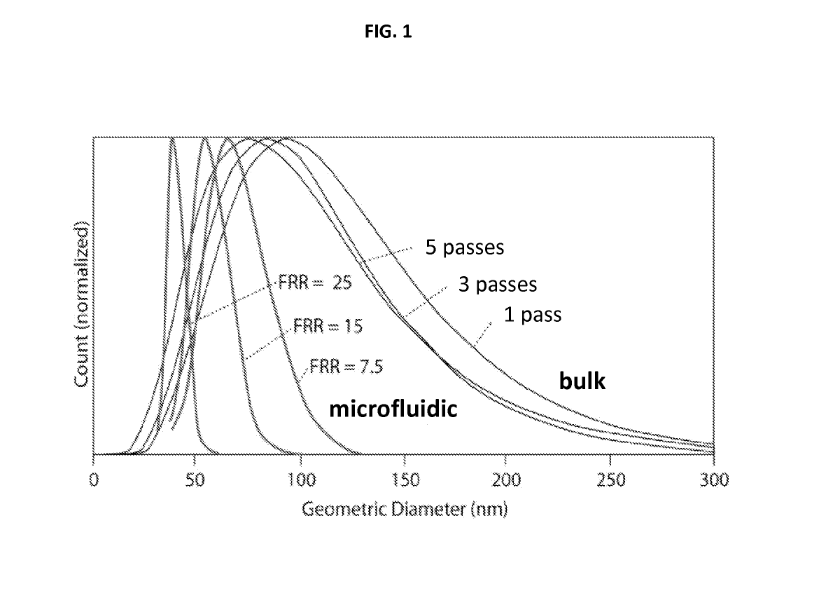

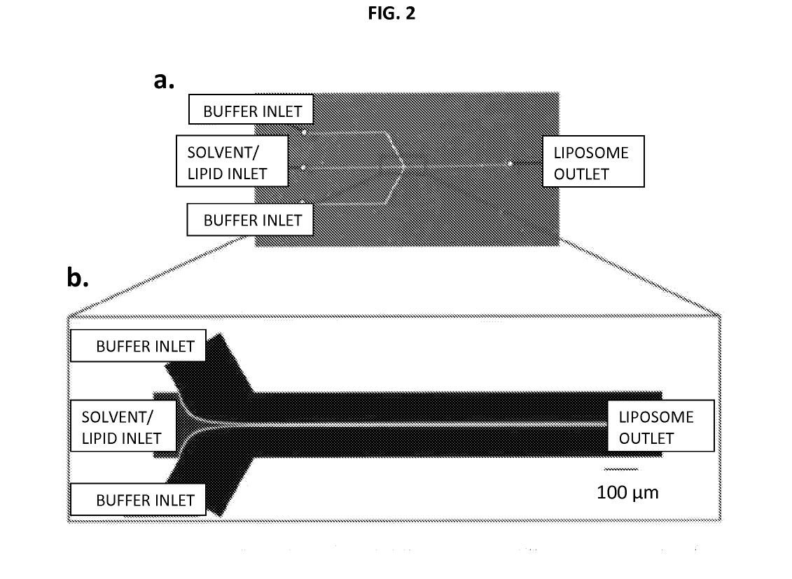

In addition, utilization of the microfluidic flow-focusing technique provides enables the production of nearly-monodisperse liposome vesicles within a range of sizes not easily attainable through other prevailing methods (approximately 40 nm and below). Preferably, the liposome populations utilized with the methods and compositions of the present invention exhibit relatively low polydispersity corresponding to relative standard deviations in nanoparticle diameter below 20%, or below 10%, or below 5%, or most preferably with low polydispersity corresponding to relative standard deviations in nanoparticle diameter below 2%. Microfluidic techniques for generating liposome encapsulated nanoparticle populations suitable for use with the present invention are disclosed by Hood et al. (2013) “Microfluidic Synthesis of PEG- and Folate-Conjugated Liposomes for One-Step Formation of Targeted Stealth Nanocarriers,” Pharm. Res. 30:1597-1607; see also Jahn et al. (2004) “Controlled vesicle self-assembly in microfluidic channels with hydrodynamic focusing,” J. Am. Chem. Soc. 126(9):2674-2675; Jahn et al. (2007) “Microfluidic Directed Formation of Liposomes of Controlled Size,” Langmuir 23:6289-6293; Jahn et al. (2008) “Preparation of nanoparticles by continuous-flow microfluidics,” J. Nanopart. Res. 10:925-934; Jahn et al. (2010) “Microfluidic Mixing and the Formation of Nanoscale Lipid Vesicles,” ACS Nano 4(4):2077-2087. Such microfluidic techniques enable the production of unilamellar lipid vesicles of tunable size within the requisite target size range to passively traverse dermal tissue, and with much lower levels of polydispersity than other synthesis methods (see FIG. 1).

An exemplary microfluidic synthesis process for producing liposome nanoparticles suitable for use with the methods and compositions of the present invention is shown in FIG. 2. A planar chip (FIG. 2(a)) containing sealed microchannels is provided, in which a lipid-containing solvent stream is hydrodynamically focused by a sheath flow of aqueous buffer within a continuous flow process (FIG. 2(b)). During the focusing step, water-soluble organic solvent, such as isopropanol or ethanol in which lipids are dissolved, diffuses into the surrounding buffer, thereby encouraging self-assembly of intermediate lipid structures, and then ultimately unilamellar liposomes downstream of the focusing zone. This approach provides the ability to generate well-defined populations of exceptionally small and nearly monodisperse liposome populations suitable for use with the disclosed methods and compositions.

The microfluidic structure and process may be adjusted in order to produce a population of liposomes having a specific target size suitable for transdermal delivery (e.g., diameters of between about 30 nm and 40 nm). See Hood et al. (2013), supra, Pharm. Res. 30:1597-1607; Jahn et al. (2007), supra, Langmuir 23:6289-6293; Jahn et al. (2008), supra, J. Nanopart. Res. 10:925-934; Jahn et al. (2010), supra, ACS Nano 4(4):2077-2087. Liposome vesicle size may be selected and tuned by adjusting the relative flow rates of the solvent and buffer streams. In addition, water soluble drugs may be readily incorporated into the liposome core or vesicles during microfluidic synthesis by adding a set of tertiary microchannels configured to inject the drug(s) between the lipid stream and outer buffer sheath flow (Id.). At the same time, lipophilic drugs may also or alternatively be incorporated by adding the drug(s) to the initial lipid solution.

Conventional bulk preparation methods for producing liposomes require significant manual handling, with multiple sequential processing steps. The resulting populations include relatively large liposomes (e.g., greater than 60 nm) and wide size distributions. Such bulk preparation methods therefore require significant infrastructure and processing, including membrane filtration to reduce the polydispersity of the final liposome populations. Even after multiple serial filtration steps, the resulting liposome populations achieved via bulk preparation methods exhibit relatively large variations in diameter. As a result, prior studies have not shown extensive penetration of traditional liposomes past the SC (Du Plessis et al. (1994), supra, Int. J. Pharm. 103:277-282; Prow et al. (2011), supra, Adv. Drug Deliv. Rev. 63:470-491; Sudhakar et al. (2012), supra, Sebastian M, Ninan N, Haghi A K, editors, Nanomedicine and Drug Delivery, Apple Academic Press, pp. 1-15; Sentjurc et al. (1999), supra, J. Control Release 59:87-97).

The liposomes utilized in accordance with the disclosed methods herein, preferably produced via microfluidic techniques, are both smaller and more narrowly distributed in diameter compared to liposomes used in prior transdermal drug delivery studies (particularly liposomes produced via bulk preparation methods). The use of smaller liposomes is significant due to the discovery that liposomes exhibit size-dependent dermal transport, with vesicles smaller than approximately 40 nm in diameter traversing the SC more effectively than larger nanoparticles (e.g., greater than 60 nm). Similarly, the low polydispersity is significant since the total fluorescence signal from a vesicle population with a wide size distribution is biased by the presence of significant number of liposomes above the mean diameter, which prohibits accurate evaluation of transport as a function of vesicle size.

Embodiments of the present invention provide for the selection of liposome size range and formulations based on the desired delivery method and/or the desired rate of drug release, which may be tailored to a specific patient care situation. In some formulations, two or more populations of liposome encapsulated drugs are provided, whereby each liposome population possesses nanoparticles having a different average diameter. Further, formulations may include a first population of liposomes encapsulating a first anesthetic, analgesic, antibiotic, hormone, or therapeutic drug, and a second population of liposomes encapsulating a second anesthetic, analgesic, antibiotic, hormone, or therapeutic drug. Additional (i.e., third, fourth, fifth, etc.) populations of liposomes encapsulating additional therapeutic or diagnostic agents may also be provided in formulations.

The characteristics of the liposome nanoparticles within a first population may be selected for effectuating or controlling a first delivery route and/or release profile, while the characteristics of the liposome nanoparticles within a second population may be selected for effectuating or controlling a second delivery route and/or release profile. For example, the average diameter of liposome nanoparticles in the first population may be sufficiently small (e.g., less than 50 nm, preferably less than about 40 nm, and preferably between about 20 nm and about 40 nm) for traversing the SC and thus for transdermal systemic drug delivery of the encapsulated drug or cargo of the liposomes of the first population. The average diameter of the liposome nanoparticles in the second population may be sufficiently large (e.g., greater than about 50 nm) for providing local drug delivery. Alternatively or additionally, the second or other population of liposome nanoparticles may have a selected size (e.g., between about 40 nm and about 50 nm, between about 30 nm and about 40 nm, etc.) that is capable of more slowly traversing the SC (as compared to smaller nanoparticles) at a desired rate, or that is capable of absorbing into only the outer dermal layers at a desired rate. Thus, by controlling the size of the nanoparticles within a particular formulation, the reach of drug delivery may be selected and controlled. A first drug or agent may be administered and maintained at a local level by utilizing nanoparticles having selected first size and formulation characteristics (for a particular delivery route and release profile), while a second drug or agent may be administered for systemic delivery by utilizing nanoparticles having selected second size and formulation characteristics (for another different delivery route and release profile). This ability to selectively control the depth of penetration of a particular drug or agent is significant in that pharmaceutical compositions with dual or multi delivery and release profiles may be provided. For example, a pharmaceutical compositions providing for local transdermal delivery of anesthetics and transdermal systemic delivery of another therapeutic agent is significant. Thus, the disclosed methods and pharmaceutical compositions allow for the creation of numerous different formulations.

The characteristics of liposome nanoparticles within a population(s) may also be selected to effectuate a continuous drug release profile. In one implementation, formulations may include nanoparticles having a range of sizes. A first group or population of nanoparticles having a first diameter size may absorb relatively quickly for immediate drug systemic or local release; a second group or population of nanoparticles having a second diameter size may absorb at a slower rate for later drug release; a third group or population of nanoparticles having a third diameter size may absorb at the slowest rate; etc. Moreover, each of the liposome groups or populations may be synthesized to release their encapsulated drug at a specific rate. The control of such release profiles via the utilization of variously sized and configured nanoparticles allows for continuous drug delivery over a relatively long period (e.g., 4 hours, 6 hours, 12, hours, 24 hours, etc.). Formulations according to embodiments of the present invention may include liposomes encapsulating an anesthetic, an analgesic, an antibiotic, a hormone, and/or some other therapeutic or diagnostic agent. Such formulations may be selectively engineered to provide a continuous and sustained release profile over a relatively long period, as described above.

By utilizing liposomes with relatively small diameters (e.g., approximately 40 nm or less), efficient transport of intact drug-laden liposomes through the SC is achieved, enabling controlled delivery rates over extended periods of time of both hydrophilic and lipophilic compounds with a high drug:lipid ratio. Thus, various treatments benefit from the utilization of drug-encapsulated liposome nanoparticles with specific average particle diameters and relatively low polydispersity.

A wide range of different drugs and formulations may be provided utilizing relatively small liposome nanoparticles with specific average sizes, and in populations exhibiting very narrow size distributions. For example, a local anesthetic formulation according to an embodiment of the present invention comprises liposomal encapsulated lidocaine. Lidocaine is a highly effective anesthetic. However, topical application of lidocaine has previously been limited due to its relatively slow uptake across the dermis. Such limitations have been overcome by the present invention by providing for relatively small liposomes, preferably having diameters of about 40 nm or less, more preferably providing for a relatively monodisperse population of liposomes having diameters of between about 30 nm and about 40 nm.

A local anesthetic formulation according to another embodiment comprises liposomal encapsulated dexmedetomidine. Dexmedetomidine has been used for general sedation and analgesia via systemic intravenous administration. Dexmedetomidine is a highly selective alpha-2 adrenergic receptor agonist increasingly used in the intensive care and operation room environments for sedation and analgesia. It has many advantages over other sedatives, including prevention of respiratory depression, conscious awareness, cooperative sedation and opioid sparing effect.

Dexmedetomidine has an additional action on peripheral nerve receptors. It has been used off-label as an adjuvant to improve anesthesia quality and duration, for epidural and peripheral nerve blocks and central nerve blocks. It has also been shown to increase anesthesia duration and analgesia time length when administered with local anesthetics. However, dexmedetomidine has a relatively short half-life (around 6 minutes), which requires the drug to be administered by pump in a continuous, intravenous infusion for sedation and analgesia.

Besides the action on alfa-2 adrenergic receptors, dexmedetomidine is an antagonist for HCN receptor (hyperpolarization-activated cation non-selective) channels. HCN channels are expressed in pain pathways at all levels in the neuraxis, from peripheral sensory organelles to thalamic and cortical neurons. The high expression level of HCN in (large diameter) sensory nerves suggests that these channels play an important role in sensory physiology. The analgesic effect of peripheral perineural dexmedetomidine was shown to be caused by enhancement of the hyperpolarization-activated cation current (Ih current), which prevents the nerve from returning from a hyperpolarized state to resting membrane potential for subsequent firing. These receptors are abundantly expressed in Aβ and Aσ nerve fibers. Aβ fibers are normally associated with non-painful light touch sensation, vibration and proprioceptive senses, but they are associated with transmission of allodynia. In neuropathic pain, unlike acute pain, unmyelinated fibers, C fibers, although classically considered “nociceptive,” may play a limited role. Dexmedetomidine acting in these receptors would thus be suitable for providing effective pain relief for this painful and poorly managed condition. However, some side effects of dexmedetomidine are a concern that currently limits its use in peripheral and central blocks as well topical application. Dexmedetomidine can decrease heart rate and decrease blood pressure, because it decreases the sympathetic tone.

Based on that, and evidence of dexmedetomidine effects in peripheral blocks, topical dexmedetomidine formulations are provided as adjuvants to local anesthetic for transdermal anesthesia and analgesia. These receptors have a high expression level in large diameter myelinated fibers (Aβ fibers), which is believed to have an important role in neuropathic pain after trauma and due to peripheral nerve injury and Spontaneous pain, and pain that is triggered by stimuli that are normally non-noxious (allodynia).

Methods and compositions of the present invention overcome such disadvantages by utilizing nearly monodisperse populations of relatively small, liposome encapsulated dexmedetomidine. The utilization of liposomes with specifically designed size ranges suitable for transdermal delivery (as discussed herein) achieves the desired clinical outcome, and also provides for a controlled rate of drug release and selected duration. The disclosed liposomal dexmedetomidine formulations allow for slow drug release in the blood stream and prolonged sedation effects, thus avoiding common side effects associated with dexmedetomidine, such as bradycardia and hypotension.

By encapsulating dexmedetomidine in a liposomal envelope, the resulting gel formulation may be applied directly to the skin surface, thereby allowing for needle-free sedation or drug delivery. The liposomal gel formulation penetrates the skin barrier and reaches the sensorial nerve fibers and/or blood stream, thereby providing sedation, and/or relieving allodynia and neuropathic pain. The liposome vesicles or envelopes allow for a slow release of dexmedetomidine in the patient, thereby allowing longer duration and avoiding high peak plasma concentrations. Thus, the encapsulated formulations are much more efficient, result in fewer side effects, and provide longer pain relief as compared to conventional methods of administering dexmedetomidine.

In addition to dexmedetomidine, encapsulated formulations may include a local anesthetic. The local anesthesia, coupled with dexmedetomidine, is more effective and has a longer duration as compared to the local anesthesia alone. Thus, various liposomal formulations of dexmedetomidine may be provided, which are specifically designed for each different application and administration route. For example, the formulation may be applied for a pre-sedation technique, providing anxiolytic effect before a surgical procedure and sedation before reaching the operating room. The formulation may also be topically applied for sedation of intensive care unit patients, as opposed to intravenous administration (e.g., such as conventional administration of dexmedetomidine).

The disclosed methods and compositions may be used for transdermal delivery of virtually any anesthetic, analgesic, antibiotic, hormone, and/or other agent (including therapeutic and diagnostic agents). Local anesthetics suitable for use with methods and compositions of the present invention include, but are not limited to, lidocaine, esteroids, ropivacaine, bupivacaine, levobupivacaine, prilocaine, procaine, tetracaine, benzocaine, chloroprocaine, mesocaine, propanocaine, ciprocaine and butacaine. Analgesics suitable for use with the present invention include, but are not limited to, glucocorticosteroids (e.g., including dexamethasone, hydrocortisone, cortisone, betamethasone, methylprednisone, methylprednisolone, prednisolone, triamcinolone, fludrocortisone, fluocinolone acetonide, fluociononide, fluorometholone and pharmaceutically acceptable mixtures thereof and salts thereof), dexmedetomidine, clonidine, non-steroidal anti-inflammatory drugs (NSAID, e.g., including Ibuprofen, Naproxen, ketoprofen, Dexibuprofen, Dexketoprofen, and all the Propionic Acid derivatives; and Indomethacin, Diclofenac, Ketorolac, Sulindac and all Acetic Acid derivatives; and Piroxicam, Meloxicam, Tenoxicam, Droxicam and all Enolic acid (Oxic am) derivatives; and Fenamic Acid derivatives and acetaminophen), and opioids (e.g., including morphine, fentanyl, alfentanil, sufentanil, methadone, nalbuphine, codeine, hydromorphone, hydrocodone, oxymorphone, oxycodone and buprenorphine). Hormones suitable for use with the present invention include, but are not limited to, peptides such as prolactin, adrenocorticoptropic hormone (ACTH), growth hormone, vasopressin, glucagon, insulin, somatostatin, cholecystokinin, gastrin, and adrenaline. Antibiotics suitable for use with the present invention include, but art not limited to, aminoglycosides, ansamycins, carbacephems, carbapenems, cephalosporins, glycopeptides, lincosamides, lipopeptides, macrolides, monobactams, nitrofurans, penicillins, quinolones, sulfonamides, tetracyclines, and polypeptide antibiotics.

The liposome encapsulated drug formulations are suitable for use in various applications and treatments. In one implementation, the disclosed liposomal drugs are utilized as a transdermal local analgesia for skin lesions and/or for relieving pain in patients, and in particular neuropathic pain. In other implementations, the disclosed liposomal drugs are suitable for use for pre-anesthetic sedation and/or for sedation of patients by applying the drug topically. By creating a depot area inside of the dermis, the encapsulated drug is able to reach the blood stream and promote adequate sedation.

In other implementations, the disclosed liposomal drugs are suitable for use as peripheral and central nerve blocks. For example, the liposomal formulations may be utilized as an adjuvant to provide longer duration of analgesia and/or anesthesia when added to local anesthetic in peripheral nerve blocks and epidurals. The drug may include nanoparticles of differing size ranges, with unilamellar or multilamellar envelopes, so long as the size and characteristics are suitable for transdermal delivery.

Encapsulated drugs in different formulations allow for better routes of administration, exhibit relatively slow release, and decreased side effects as compared to conventional drugs. Each application may utilize a selected liposomal envelope size and composition that enables a better safety profile and improved drug efficacy.

Having generally described the invention, the same will be further understood through reference to the following additional example, which is provided by way of illustration and not intended to be limiting of the present invention unless specified.

### Example 1

Continuous-flow microfluidic liposome synthesis was utilized to generate small, nearly monodisperse lipid vesicles within a target size range of interest suitable for transdermal drug delivery to demonstrate size-dependent passive uptake of liposomes into ex vivo porcine dermal tissue.

Materials and Methods

Device Fabrication

Microfluidic devices made of polydimethylsiloxane (PDMS) and glass were fabricated using soft lithographic methods. Briefly, SU-8 (negative photoresist) was spin coated onto a 10 cm silicon wafer, patterned via ultraviolet light against a photomask bearing the designs for the desired fluidic channels (50/μm wide, 300 μm tall). The patterned silicon wafer was then developed and used as a mold for PDMS. Once cured, the PDMS was removed from the mold and exposed with glass to oxygen plasma. The final device was created by pressing the two pieces, forming a permanent bond. The PDMS-glass microfluidic devices were used to form small, nearly monodisperse liposomes using previously demonstrated methods (see Jahn et al. (2004), supra, J. Am. Chem. Soc. 126:2674-2675; Jahn et al. (2007), supra, Langmuir 23:6289-6293; Jahn et al. (2010), supra, ACS Nano 4(4):2077-2087).

Lipid Mixture and Hydration Buffer Preparation

Dimyristoylphosphatidylcholine (DMPC), cholesterol, and phosphoethanolamine-[methoxy(polyethylene glycol)-2000] (pEG(2000)-PE) were mixed in chloroform in molar ratio 75:25:5 and then placed in a vacuum desiccator for at least 24 hours to allow complete solvent removal. PEG-lipid enables the formation of smaller vesicles, provides steric stabilization, and serves as a protective shield from the immune system for liposomes should any reach the vasculature. The dried lipid mixtures were then re-dissolved in anhydrous ethanol containing 1 wt % of a lipophilic membrane dye (1,1′-dioctadecyl-3,3,3′,3′-tetramethylindocarbocyanine perchlorate; DiI-C18; DiI) for a total lipid concentration of 40 mM. A 10 mM phosphate buffered saline (PBS) solution at pH 7.4 was used as a hydration buffer. All fluids (solvent and buffer) were filtered through 0.22 μm filters before being introduced to the microfluidic device.

Microfluidic Liposome Synthesis and Characterization

Liposomes were prepared by injecting the lipid-solvent mixture between two buffer inputs into the PDMS glass micro fluidic device (see FIG. 2). The flow rate ratio (FRR), which is defined as the volumetric flow rate of buffer to that of the solvent, was set to FRR 4 and FRR 50 for two populations of liposomes above and below the size range expected to passively traverse the dermal layer. Linear flow velocity of the total flow for all FRRs was kept constant (0,125 m/s) for a volumetric flow rate of 112 μl/min. The microfluidic device was operated on a hot plate at 50° C. throughout the entire procedure to facilitate the formation of small vesicles (see Zook et al. (2010) “Effects of temperature, acyl chain length, and flow-rate ratio on liposome formation and size in a microfluidic hydrodynamic focusing device,” Soft Matter 6(6):1352). The liposome populations were characterized for size using a Malvern Zetasizer Nano ZSP.

The vesicles produced contained DiI in their bilayer to assist in fluorescence imaging, which helped assess the depth of penetration. To remove any remaining dye not incorporated into the liposomes during the formation process, the samples were purified via size exclusion chromatography on Sephadex G-25 (PD-10) columns that were equilibrated with PBS.

Ex Vivo Application of Liposomes to Porcine Tissue

Porcine tissue was used to evaluate passive transdermal diffusion of the microfluidic-synthesized liposomes due to its morphological and functional similarities to human skin (see Kong et al. (2011) “Characterization of porcine skin as a model for human skin studies using infrared spectroscopic imaging,” The Analyst 136(142359-66). A Yorkshire pig (4 weeks, 5 kg), which was being sacrificed for another study, was used for these experiments. One ear from the pig was removed immediately following general anesthesia and used immediately. Liposome solutions (50 μL) each size population (32.4 nm and 287.1 nm) were applied to different locations on the outer side of the ear. After 15 minutes, the ears containing liposomal dye were removed and immediately placed in a freezer in covered petri dishes.

Cryosectioning and Fluorescence Imaging

Subsequent to liposome exposure, the removed ears were sliced (using a fresh razor) perpendicularly to the skin surface and directly through the sections where liposome-containing solutions were applied. The tissue samples were then embedded into cryo-OCT media and placed in a freezer (−80° C.). Once frozen, the embedded tissues were mounted in a cryostat microtome (HM550 series, Richard Allen Scientific) for slicing. Starting from the initial coarse cut, a few hundred microns of tissue was sliced off and discarded to ensure the sections used for imaging were distant from the blade-cut region. After the initial segment was removed, smaller sections (30 μm) were sliced and placed onto gelatin-treated glass slides. The sections were immediately imaged using an inverted epifluorescence microscope (Nikon TE-2000 S). Bright field and fluorescence images at a 528 nm-553 nm range excitation wavelength (green filter) were taken and overlaid to assess and validate the depth of liposome penetration into the dermal tissues.

Results and Discussion

Microfluidic Liposome Synthesis

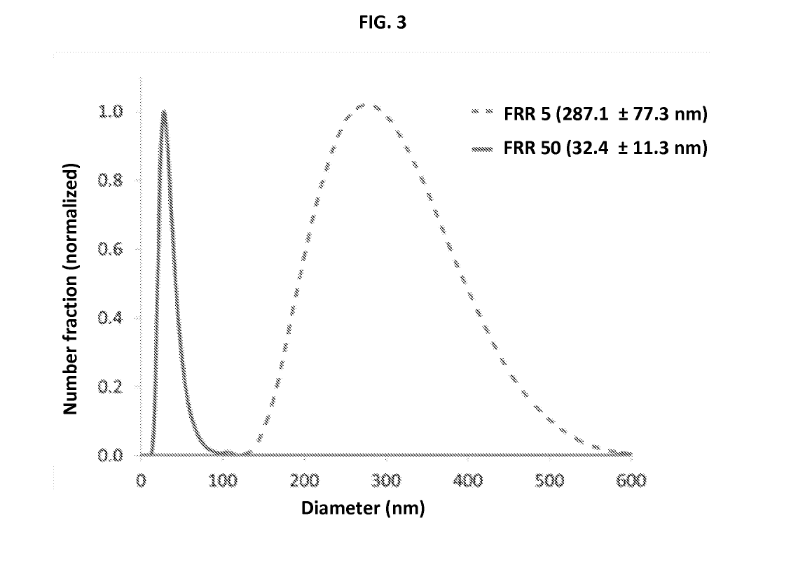

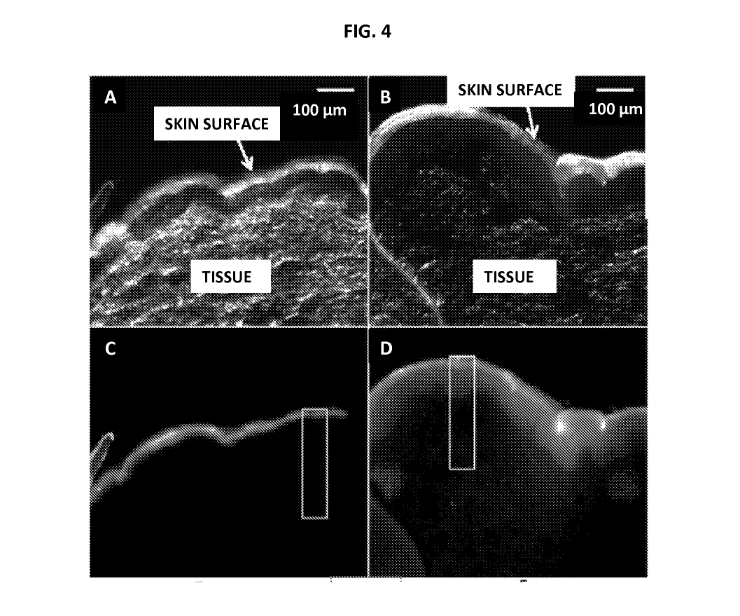

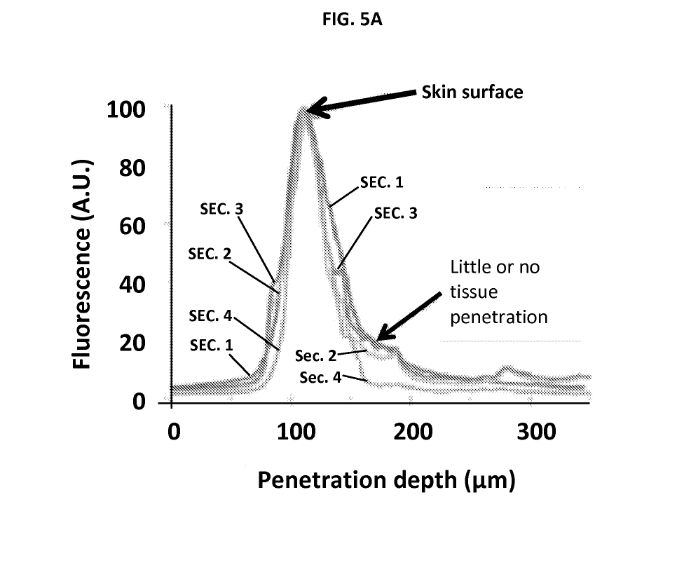

Continuous-flow microfluidic production of liposomes provided populations of vesicles near the size range of particles observed to transport through porous tissues (FIG. 3). The liposomes, which were on the order of size necessary to traverse skin layers (32.4±11.3 nm), were formed with remarkably low levels of polydispersity. Larger liposomes (287.1±77.3 nm) of identical composition were also formed for comparison.

Transdermal Liposome Penetration

Cryomicrotoming and fluorescence microscopy of the exposed tissue samples revealed passive uptake of dye-laden, smaller liposomes (32.4 nm) into the porcine tissue as well as exclusion of liposomes with identical composition but larger diameters (287.1 nm) (FIG. 4). The bright field/fluorescence overlay images of the porcine skin exposed to larger 287.1 nm and smaller 32.4 nm liposomes (FIG. 4 (A) and FIG. 4 (B), respectively) show the similar morphology of the porcine dermis across samples with an improved penetration of the smaller liposomes through the dermal layers. The single channel fluorescence images of the larger 287.1 nm and smaller 32.4 nm liposomes (FIG. 4(C) and FIG. 4(D), respectively) further divulge the increased permeation depth of the smaller liposomes through the tissue.

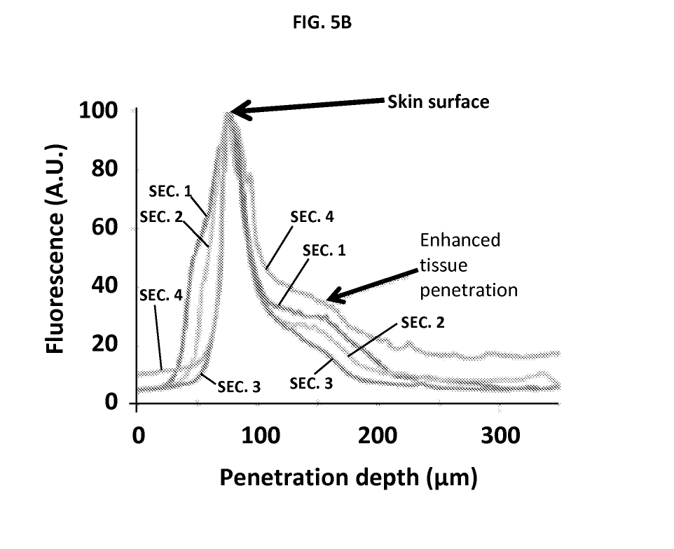

Fluorescence intensity profiles through the tissues over multiple images revealed a more quantitative depiction of the enhanced dye penetration for the smaller liposome samples as compared to the larger liposome samples (FIG. 5(A) and FIG. 5(B)). It is evident that fluorescence signal is seen much deeper in the tissue with the smaller 32.4 nm liposomes, while the larger 278.1 nm liposomes appear to only show fluorescence at the uppermost layer of skin. The small, highly-monodisperse 32.4 nm liposomes are on the size scale of intracellular junctions and other sites which present openings in the skin, which accounts for their ability to passively enter the SC and diffuse through the skin layers, while the larger 287.1 nm liposomes simply remain on the top layer of the skin. Thus, these results are in stark contrast to other techniques, in which larger, more polydisperse liposomes do not penetrate the SC.

The results herein validate the selective production and utilization of liposome encapsulated nanoparticles having small yet distinct sizes which may be passively incorporated into dermal tissue. The use of liposomes below a target size limit traverses the dermal layer without the need for additional treatment, thereby enabling the use of liposomes for transdermal drug delivery applications previously unattainable. Novel formulations which will quickly penetrate through the skin layers are achieved, promoting fast drug onset while also providing long duration and improved safety profiles.

Thus, the disclosed microfluidic methods of liposome synthesis may be exploited to produce populations of liposomes with small incremental size differences to optimize formulations for dermal uptake. The resulting liposome encapsulated formulations are suitable for a variety of applications, including in clinical anesthesia and pain medicine. By controlling liposome population size and characteristics, transdermal uptake for topical applications and for systemic drug delivery is achieved.

### Example 2

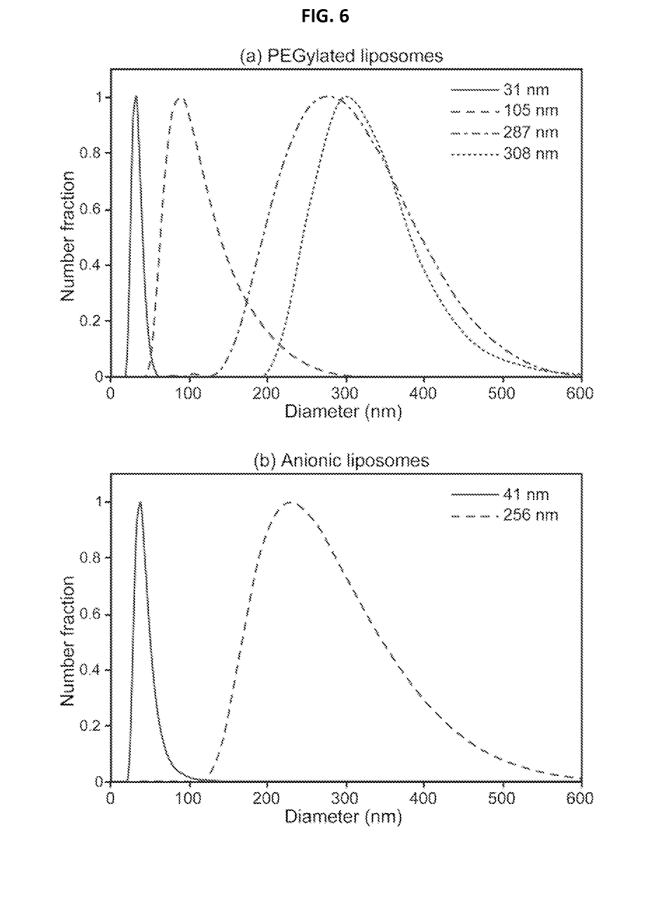

Microfluidic synthesis of small and nearly-monodisperse liposomes was used to further investigate size-dependent passive transdermal transport of nanoscale lipid vesicles. In this further study, microfluidic-enabled liposome preparations with mean diameters ranging from 31 nm to 308 nm were prepared (FIG. 6). Within this size range, two classes of liposomes were formed that differed by the incorporation of small amounts of either anionic lipids or PEGylated lipids, enabling the influence of surface chemistry on trans-SC flux to be investigated. For liposome preparations, a polydispersity index of the microfluidic-synthesized liposomes ranged from 0.035 to 0.135; as a comparison, a previous study investigating vesicles as small as 120 nm reported the use of liposomes with polydispersity indices varying from 0.1 to 0.3 (Verma et al. (2003) “Particle size of liposomes influences dermal delivery of substances into skin,” Int. J. Pharm, 258:141-151).

As expected, large liposomes with diameters (e.g., above 105 nm) were found to be excluded from deeper skin layers past the SC, the primary barrier to nanoparticle transport, while liposomes with smaller mean diameters between (31 nm to 41 nm) exhibited significantly enhanced penetration. Furthermore, multicolor fluorescence imaging revealed that the smaller liposomes passed rapidly through the SC without vesicle rupture. The study further validated the discovery that nanoscale liposomes with well-controlled size and minimal size variance are excellent vehicles for transdermal delivery of functional nanoparticle drugs.

Materials and Methods

Lipid Mixture and Hydration Buffer Preparation

Two variations of lipid mixtures were prepared to analyze the resulting penetration depth of both PEG-conjugated (PEGylated) and negatively-charged (anionic) into dermal tissue. In addition to enhancing liposome stability, PEG may be attached to the exterior of liposomes as a protective shield from the immune system during blood circulation (e.g., see Immordino et al. (2006) “Stealth liposomes: review of the basic science, rationale, and clinical applications, existing and potential,” Int. J. Nanomedicine 1:297-315), increasing the bioavailability of PEGylated liposomes that are able to reach and enter subcutaneous capillaries after transdermal transport.

For PEGylated liposomes, dimyristoylphosphatidylcholine (DMPC), cholesterol, and dipalmitoylphosphatidylethanolamine-PEG 2000 (PEG2000-PE) (Avanti Polar Lipids Inc., Alabaster, Ala.) were combined in chloroform (Mallinckrodt Baker Inc., Phillipsburg, N.J.) at a molar ratio of 70:25:5. For anionic liposomes, DMPC, cholesterol (both from Avanti), and anionic surfactant dihexadecyl phosphate (DCP) (Sigma Aldrich, St. Louis, Mo.) were mixed in chloroform (Mallinckrodt Baker Inc.) at a molar ratio of 50:40:10. The lipid mixtures were prepared in glass scintillation vials then stored in a vacuum desiccator for at least 24 hours for complete solvent removal. The desiccated lipid mixtures were re-dissolved in anhydrous ethanol (Sigma Aldrich) for a total lipid concentration of 40 mM. To assist in fluorescent imaging, a lipophilic membrane dye, 1,1′-dioctadecyl-3,3,3′,3′-tetramethylindocarbocyanine perchlorate (DiI-C₁₈; DiI) (Life Technologies, Carlsbad, Calif.) was included into the lipid mixtures (1 wt %). A 10 mM phosphate buffered saline (PBS) (Sigma Aldrich) solution at pH 7.4 was used as a hydration buffer, with selected samples containing 1 mM hydrophilic sodium fluorescein salt (SF) (Sigma Aldrich) as a hydrophilic dye. All fluids (solvent and buffer) were passed through 0.22 μm filters (Millipore Corp., New Bedford, Mass.) before being introduced to the microfluidic device.

Liposome Synthesis and Characterization

PEGylated and anionic liposomes were prepared using microfluidic techniques as noted above (see Jahn et al. (2004), supra, J. Am. Chem. Soc. 126:2674-2675; Jahn et al. (2007), supra, Langmuir 23:6289-6293; Jahn et al. (2010), supra, ACS Nano 4(4):2077-2087; see also Hood et al. (2013), supra, Pharm. Res. 30:1597-1607). Briefly, a flow-focusing microchannel network for liposome synthesis was fabricated (see Hood et al. (2013) “Microfluidic synthesis of PEGylated and folate receptor-targeted liposomes,” Pharm. Res. in press. doi:10.1007/s11095-013-0998-3). All microchannels in the final device were nominally 50 μm wide and 300 μm tall. The prepared lipid-ethanol solution was injected into the microfluidic device between two sheath flows of the aqueous buffer. The flow rate ratio (FRR), defined as the ratio of the volumetric flow rate of the aqueous buffer to the volumetric flow rate of the ethanol, was varied between 5-50 to produce liposomes with modal diameters ranging from about 31 nm to about 308 nm. Total average linear flow velocity for all FRRs was kept constant (0.125 m/s) for a total volumetric flow rate of 112 μL/min. To enable the formation of smaller vesicles, the temperature of the microfluidic device was controlled by contacting the glass slide of the device with a hot plate at 50° C. throughout the entire synthesis process. The resulting liposome populations were characterized for size via dynamic light scattering (Nano ZSP, Malvern Instruments Ltd., UK). Size distribution plots were generated by fitting spline curves to the binned distribution data imported from the dynamic light scattering instrument.

The microfluidic-generated liposomes contained lipophilic DiI in their bilayers and hydrophilic SF in their cores to enable fluorescence imaging of tissue penetration depth. To remove any remaining dye not incorporated into the liposomes during the synthesis process, all liposome samples were purified via size exclusion chromatography on Sephadex G-25 PD-10 columns (GE Healthcare, Piscataway, N.J.) equilibrated with PBS immediately before application to the tissue. Gel filtration using the PD-10 columns provides efficient buffer exchange for removal of ethanol used in the liposome formation process, thereby preventing variations in ethanol concentration (2-16%) used for different liposome populations from affecting skin permeation experiments. Final lipid concentrations following gel filtration ranged from 0.56 mM to 4.76 mM, depending on the FRR used for liposome synthesis.

Tissue Exposure and Cryosectioning

Porcine ear tissue from Yorkshire piglets (4 weeks, 5 kg) was selected due to its morphological and functional resemblance to human skin. Porcine ear skin in vitro has shown remarkably similar biophysical properties to human skin in vivo, particularly in terms of the diffusivity and permeability coefficient of water across the SC (Sekkat et al. (2002) “Biophysical study of porcine ear skin in vitro and its comparison to human skin in vivo,” J. Pharm. Sci. 91:2376-2381). Studies have also indicated that porcine skin is extremely similar both structurally and chemically to its human counterpart, exhibits chemical properties which are rather consistent across different samples and stable over time at room temperature, therefore porcine skin is a valuable tool for investigating diffusion dynamics of materials with human skin (Kong et al. (2011) “Characterization of porcine skin as a model for human skin studies using infrared spectroscopic imaging,” Analyst 136:2359-2366).

One ear from each animal was removed following general anesthesia. Liposome solutions were immediately applied in 50 μL aliquots for each size in different locations on the outside of the ear, resulting in spot areas ranging from 0.25-0.5 cm², and incubated for 15 minutes at room temperature. This exposure method was chosen over the use of a perfusion cell for focusing on short-term SC transport rather than long-term behavior of the nanoparticles within the dermis. For the characterization of size-dependent transport, all liposome solutions covering the full range of size distributions were deposited on ears from a single animal to minimize the influence of tissue morphology variations between animals. Different animals were used for each set of experiments characterizing PEGylated liposome transport, anionic liposome transport, and co-distribution of lipophilic and hydrophilic dyes.

Following incubation, the ear tissue was placed in a plastic petri dish and frozen. The frozen tissue was bulk sectioned, embedded using Tissue-Tek Cryo-OCT compound (Fisher Scientific, Pittsburgh, Pa.), and frozen at −80° C. The frozen tissue blocks were then sectioned into smaller slices, nominally 30 μm thick and revealing dermal tissues at least 300 μm from the surface, using a HM550 cryostat microtome (Richard Allan Scientific, Kalamazoo, Mich.) and placed onto gelatin-treated glass slides for imaging. Sections were procured from the tissue directly beneath each of the applied liposome volumes, with the plane of each section aligned through the center of its corresponding droplet. Sectioning was performed with the blade oriented perpendicular to the skin surface and the blade path in the direction of the SC to prevent artifacts that could result from mechanical displacement of liposomes, dye, or tissue normal to the SC layer.

Fluorescence Microscopy and Image Processing

The 30 nm thick tissue sections were imaged using a TE-2000 S inverted epifluorescence microscope (Nikon, Melville, N.Y.). Brightfield images and fluorescence images at 528 nm-553 nm (green filter; DiI) and 465 nm-495 nm (blue filter; SF) excitation wavelengths were acquired and overlaid to confirm and evaluate the extent of liposome penetration into the dermal tissue and to assess colocalization of the lipophilic and hydrophilic dyes.

ImageJ software (National Institutes of Health, Bethesda, Md.) was used to analyze the images. Fluorescence intensity profiles were extracted using 10 μm wide regions of interest (ROIs), with data from multiple ROIs combined to generate quantitative profiles of liposome penetration depth. The intensity data was averaged across 5 ROIs per sample, then normalized to peak intensity and aligned to reveal the average fluorescence signal seen within each tissue sample below the SC. Dye colocalization was analyzed using the JACoP plugin with ImageJ (Bolte et al. (2006) “A guided tour into subcellular colocalization analysis in light microscopy,” J. Microsc. 224:213-232). In all experiments, image analysis was performed independently for each dye.

Results and Discussion

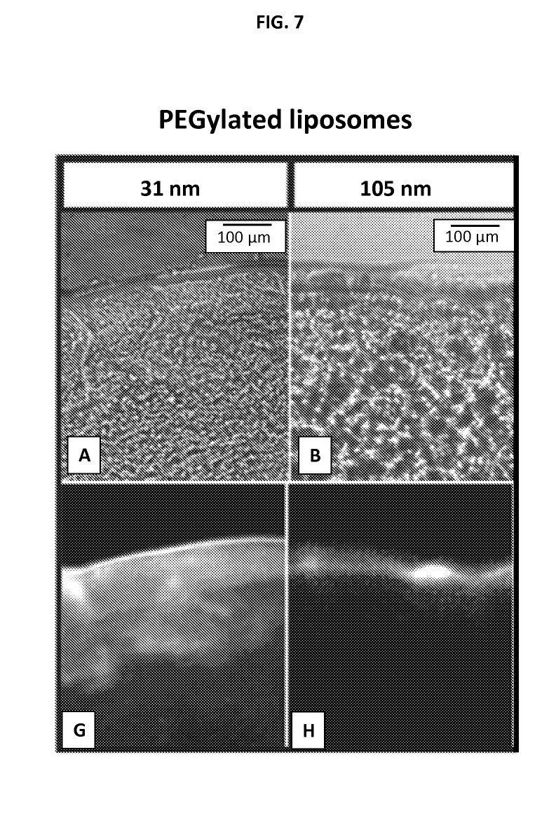

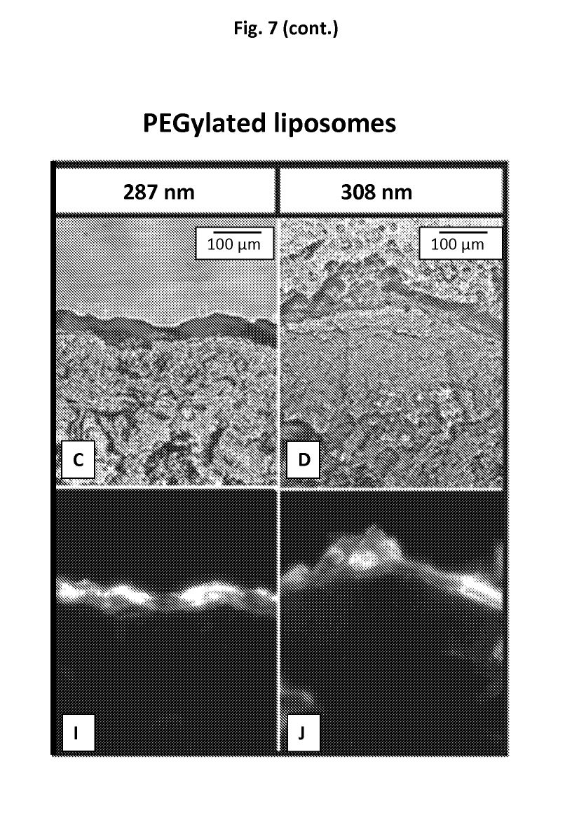

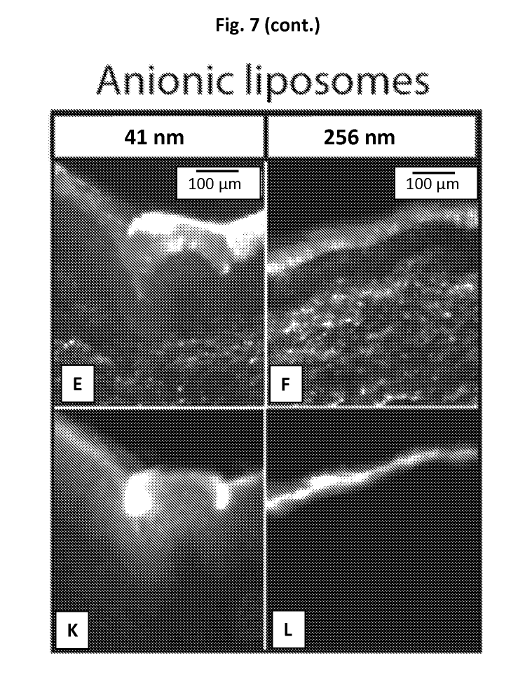

Fluorescence microscopy of microtomed porcine ear tissue after incubation with the various liposome preparations shows a marked difference in the dermal penetration of dyes between tissues exposed to either larger or smaller liposome preparations. FIG. 7 shows representative fluorescent images revealing the distribution of DiI dye within the tissue sections. Skin samples exposed to the larger 105 nm to 308 nm liposomes (PEGylated and anionic) consistently exhibit bright bands of fluorescence associated with the SC, with very little fluorescence within deeper skin layers, revealing that these larger liposomes are either physically excluded by the narrow inter-corneocyte spaces, or are ruptured in the process of traversing the SC. In the latter case, the lipids and lipophilic dyes from the ruptured liposomes are likely to adhere to or associate with surrounding cells and extra-cellular material (Elsayed et al. (2007) “Lipid vesicles for skin delivery of drugs: reviewing three decades of research,” Int. J. Pharm. 332:1-16).

Conversely, the smaller 31 nm and 41 nm liposome samples revealed a more evenly distributed dye profile throughout the skin, traversing the SC and entering the underlying layers of tissue in multiple instances with less significant accumulation in the SC (FIG. 7). Note that several bright features that appear in deeper layers within some images are believed to result from imperfections caused by vessels or voids created during cryosectioning. Skin locations with capillaries were excluded due to the known autofluorescence of whole blood between wavelengths of 450-600 nm (Gao et al. (2004) “Characteristics of blood fluorescence spectra using low-level, 457.9-nm excitation from Ar+ laser,” Chinese Opt. Left. 2:160-161) and the relatively low concentration of fluorescent molecules in the liposome samples. Regions with significant voids created by tearing of the thin tissue sections during microtoming were excluded in order to maintain consistency throughout the samples.

In control samples using free SF dye applied to the skin in liposome-free buffer, no penetration beyond the SC was observed. Dye penetration into deeper skin layers showed a strong dependence on liposome size, irrespective of charge state as determined by the presentation of PEG or anionic lipids on the vesicle surfaces. This was consistent with our initial hypothesis that dermal transport of lipid vesicles is a size-based phenomenon, and the ability of the smallest liposomes to traverse the SC and reach lower layers of skin is a direct result of the reduced liposome diameters. In some samples, bright and highly localized defects were visible in the dermis and epidermis. These features are routinely observed in dermal transport studies, and are the result of enhanced particle transport through hair-follicles, pores, and skin perforations (Rolland et al. (1993) “Site-specific drug delivery to pilosebaceous structures using polymeric microspheres,” Pharm. Res. 10:1738-1744; Mordon et al. (2003) “Site-specific methylene blue delivery to pilosebaceous structures using highly porous nylon microspheres: an experimental evaluation,” Lasers Surg. Med. 33:119-125). This uneven, defect-based liposome penetration pathway is, by nature, not highly correlated to liposome size.

The more diffuse, evenly distributed fluorescence signal seen in the epidermis in the small (31 nm and 41 nm) liposome samples is compelling evidence of liposome transport across the SC by a passive inter-corneocyte pathway (see Cevc G (2004), supra, Adv. Drug Deliv. Rev. 56:675-711; Bouwstra et al. (2003) “Structure of the skin barrier and its modulation by vesicular formulations,” Prog. Lipid Res. 42:1-36; Vogt et al. (2006), supra, J. Invest. Dermatol. 126:1316-1322; Küchler et al. (2009), supra, Eur. J. Pharm. Biopharm. Off J. Arbeitsgemeinschaft fur Pharm. Verfahrenstechnik eV 71: 243-250). For tissue samples where hair follicles were present, enhanced transport was observed for all liposomes populations; accordingly, results from these samples were omitted from analysis to prevent the confounding influence of follicular transport on analysis of SC penetration.

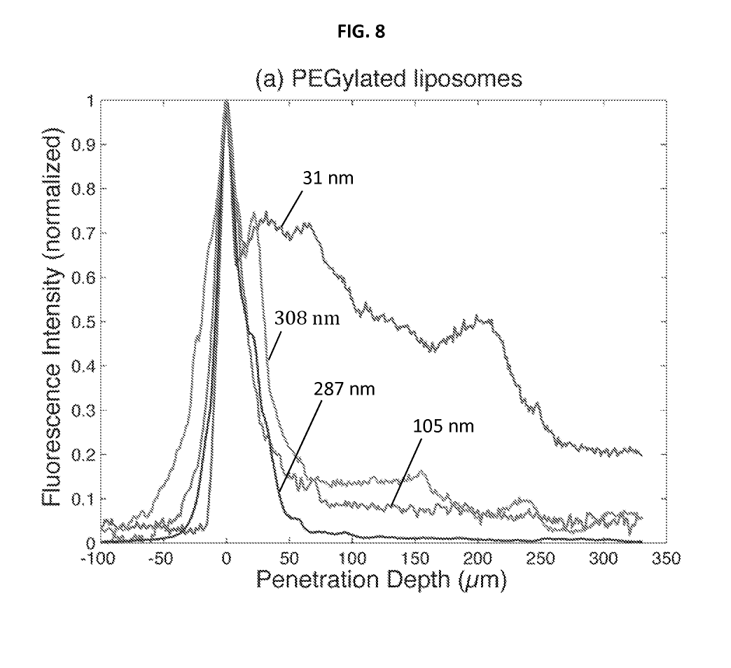

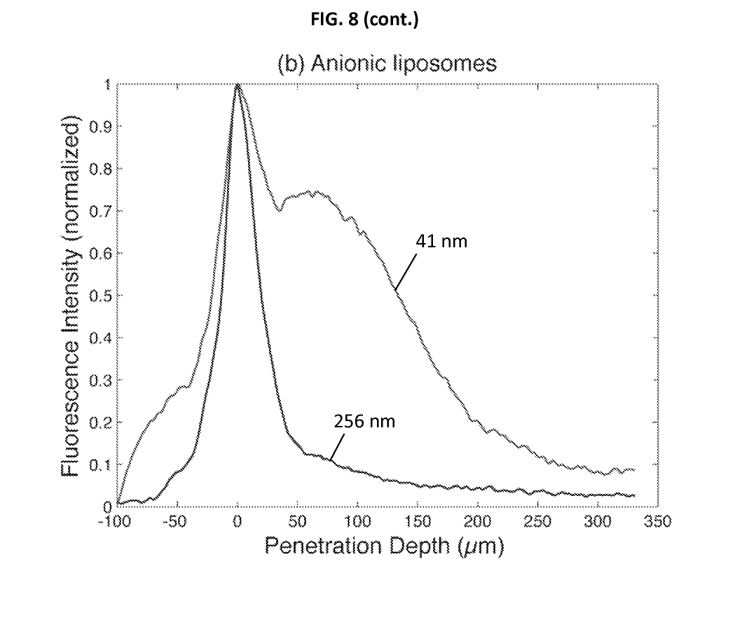

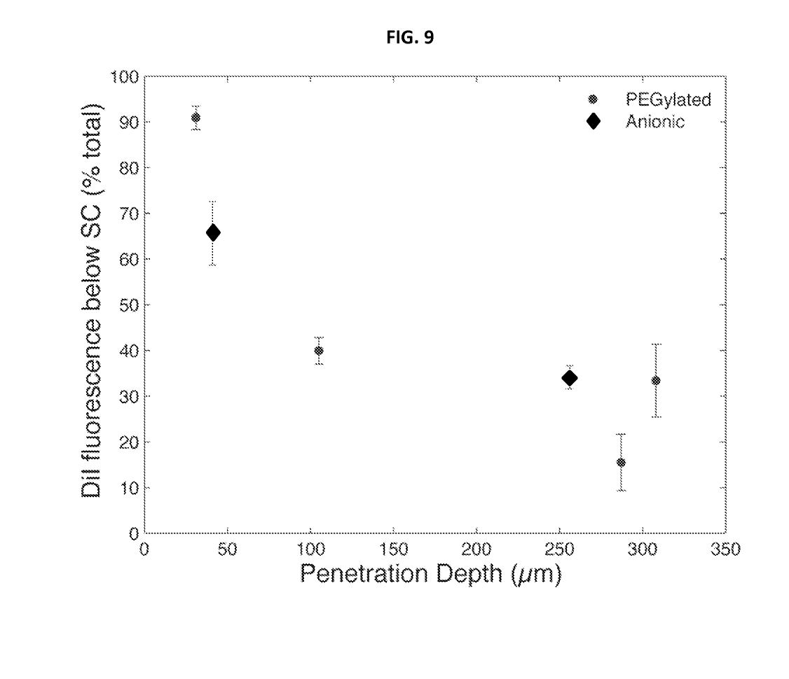

For quantitative comparison of liposome penetration, ImageJ software was used to obtain plot profiles of fluorescence intensity normal to the tissue surface. Profiles of each tissue section were averaged over five (5) representative regions per sample (FIG. 8). These profiles were normalized for maximum fluorescence intensity per profile and aligned to the midpoint of the SC, across all samples. The SC thickness was determined from averaged manual measurements using brightfield images of each tissue, ranging from 15 μm to 40 μm, which is in agreement with previously reported values for porcine skin (Jacobi et al. (2007) “Porcine ear skin: an in vitro model for human skin,” Skin Res. Technol. 13:19-24). The percentage of DiI fluorescence intensity observed beneath the SC compared to the total observed fluorescence signal was calculated from the plot profiles for each sample and compared across different liposome sizes and surface chemistries (FIG. 9). We note that this technique assumes a linear relationship between fluorescence intensity and liposome concentration, an assumption that does not hold for samples where liposomes are highly concentrated in one area causing a local saturation of fluorescence intensity, as observed in some images from the larger (diameter greater than 105 nm) liposomes tested. This saturation effect leads to systematic underreporting of liposomes trapped in the SC, and thus a bias toward higher measured penetration efficiencies for such larger liposomes can occur. Detector saturation was avoided while maintaining identical imaging conditions across all samples used. Note also that efforts were made to omit from analysis tissue sections with large voids, blood vessels, or hair follicles; some regions with anomalous fluorescent patches appeared in several images, particularly for the larger 308 nm PEGylated liposomes as seen in FIG. 7.

The small 31 nm PEGylated liposomes passed the SC in large numbers (91%), which is 590% greater than results observed for the larger 105 nm to 308 nm vesicles. The small 41 nm diameter anionic liposomes show 65% of their total DiI signal under the SC, which is 200% greater than results observed for the 256 nm diameter liposomes. Thus, both populations of smaller liposomes exhibit significantly enhanced penetration through dermal tissues compared to the larger vesicles, which is consistent with the behavior observed for other nanoparticles smaller than 50 nm in diameter, thus revealing the size-dependent transdermal transport characteristics of smaller microfluidic-enabled liposome nanoparticles.

Previous studies have failed to establish whether liposomes can traverse the SC intact. Penetration of fluorescent reporter molecules may occur as a result of liposome rupture or leakage during passage through the SC, with enhanced permeation of free dye possibly resulting from interactions between liposomes and dermal lipid structures. To explore this issue for the microfluidic-enabled liposomes having selected sizes, a combination of hydrophilic dye (SF) and lipophilic dye (DiI) were simultaneously incorporated during liposome formation into the vesicle cores and bilayers, respectively. Due to the lipid structure of the SC, diffusive transport of free hydrophilic and hydrophobic solutes is expected to vary significantly (Akomeah et al. (2007) “Variability in human skin permeability in vitro: comparing penetrants with different physicochemical properties,” J. Pharm. Sci. 96:824-834; Mitragotri S (2003) “Modeling skin permeability to hydrophilic and hydrophobic solutes based on four permeation pathways,” J. Control Release. 86:69-92), such that a lack of spatial correlation between the two dyes would imply that the liposomes had ruptured or leaked, allowing the hydrophilic dye (SF) to permeate through the tissue at a different rate than the lipophilic dye (DiI). Conversely, a high degree of spatial correlation would suggest the presence of intact vesicles.

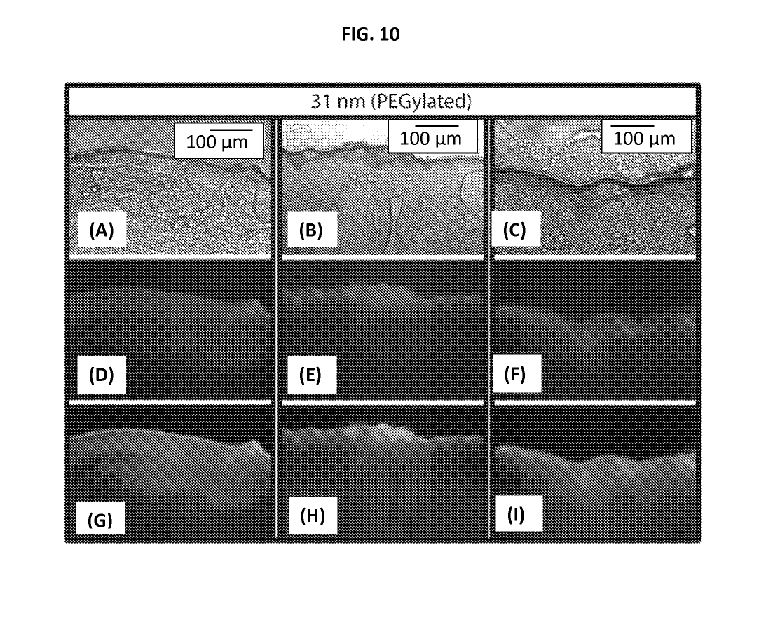

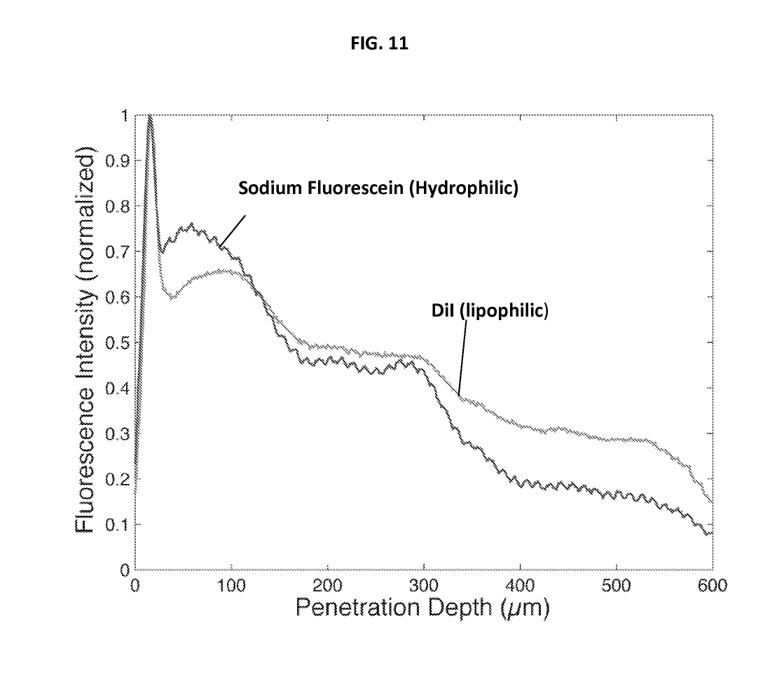

For the case of 31 nm liposomes, two color imaging of the exposed tissue sections reveals strong agreement between the distributions of hydrophilic (FIG. 10, plates G, H, I; green) and lipophilic (FIG. 10, plates D, E, F; red) signals through the SC and into the epidermis for all samples, as revealed through both the images (FIG. 10) and the dye penetration depth profiles taken through the depth of the tissue (FIG. 11). Using Pearson's correlation coefficient (p) as a measure of the degree of linear dependence between the spatial distributions of each dye, an average value of p=0.92 was determined for the 31 nm PEGylated liposomes, indicating a high degree of correlation between the dye locations. This evidence strongly indicates that the small liposomes successfully penetrate through the SC intact with minimal leakage of their cargo.

Similar experiments performed using larger liposomes resulted in measured values of ρ=0.81 and ρ=0.75 for 308 nm and 105 nm liposomes, respectively. This relatively poor correlation, together with the overall lack of significant dye penetration (FIG. 8), indicates that some degree of vesicle degradation and free dye diffusion occurred for these larger liposomes.

Concluding, a microfluidic liposome synthesis technique was leveraged to evaluate size-dependent transdermal delivery of liposomes through ex vivo porcine tissues. Compared to larger vesicles, where dye penetration across the SC is presumed to occur primarily through a combination of vesicle rupture and transport along follicular pathways, the smaller 31 nm diameter and 41 nm diameter liposomes traversed and transported their intra-liposomal contents across the full surface of the SC and into deep dermal tissues, with penetration depths of several hundred micrometers or more observed with a short 15 minute incubation.

Multicolor fluorescence imaging of hydrophilic and hydrophobic dyes incorporated into the liposomes during synthesis further revealed that the smallest (e.g., 31 nm) liposomes were able to traverse dermal layers intact, and thus would be suitable for various clinical applications requiring co-delivery of therapeutic reagents with dissimilar chemical properties, nanoparticle-mediated drug release, or transport of intact nanocarriers to the bloodstream for systemic delivery. The disclosed studies herein also represent the first demonstration of passive transdermal diffusion of nanoscale liposomes. Thus, the use of the size specific nanoparticles for effective delivery of lipophilic, hydrophilic, and amphipathic compounds to underlying dermal layers is achieved.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of transdermal drug delivery, comprising the steps of:
encapsulating an agent in liposome vesicles to form a population of encapsulated nanoparticles having an average diameter within a target size range; and
topically administering the encapsulated nanoparticles for passive transdermal delivery of the agent to a patient.

2. The method of claim 1, wherein the agent is selected from the group consisting of an anesthetic, an analgesic, an antibiotic, and a hormone.

3. The method of claim 2, wherein the anesthetic is selected from the group consisting of lidocaine, ropivacaine, bupivacaine, levobupivacaine, prilocaine, procaine, tetracaine, benzocaine, chloroprocaine, mesocaine, propanocaine, ciprocaine and butacaine.

4. The method of claim 2, wherein the analgesic is selected from the group consisting of glucocorticosteroid, dexmedetomidine, clonidine, a non-steroidal anti-inflammatory (NSAID), and an opioid.

5. The method of claim 4, wherein the glucocorticosteroid is selected from the group consisting of dexamethasone, hydrocortisone, cortisone, betamethasone, methylprednisone, methylprednisolone, prednisolone, triamcinolone, fludrocortisone, fluocinolone acetonide, fluociononide, fluorometholone and pharmaceutically acceptable mixtures thereof and salts thereof.

6. The method of claim 4, wherein the NSAID is selected from the group consisting of ibuprofen, naproxen, ketoprofen, dexibuprofen, dexketoprofen, a propionic acid derivatives, indomethacin, diclofenac, ketorolac, sulindac, an acetic acid derivative, piroxicam, meloxicam, tenoxicam, droxicam, an enolic acid (oxicam) derivative, a fenamic acid derivative, and acetaminophen.

7. The method of claim 4, wherein the opioid is selected from the group consisting of morphine, fentanyl, alfentanil, sufentanil, methadone, nalbuphine, codeine, hydromorphone, hydrocodone, oxymorphone, oxycodone and buprenorphine.

8. The method of claim 2, wherein the hormone is selected from the group consisting of prolactin, adrenocorticoptropic hormone (ACTH), growth hormone, vasopressin, glucagon, insulin, somatostatin, cholecystokinin, gastrin, and adrenaline.

9. The method of claim 2, wherein the antibiotic is selected from the group consisting of an aminoglycoside, an ansamycin, a carbacephem, a carbapenem, a cephalosporin, a glycopeptide, a lincosamide, a lipopeptide, a macrolide, a monobactam, a nitrofuran, a penicillin, a quinolone, a sulfonamide, a tetracycline, and a polypeptide antibiotic.

10. The method of claim 1, wherein the agent provides local analgesia in an area of topical application.

11. The method of claim 1, wherein the agent is transdermally delivered to the bloodstream of the patient and provides central analgesia.

12. The method of claim 1, comprising the further step of selecting the average diameter during said encapsulating step.

13. The method of claim 12, wherein the average diameter is selected based on a target drug delivery and release profile.

14. The method of claim 1, wherein the population of encapsulated nanoparticles has a polydispersity in nanoparticle diameter of less than about 20%.

15. The method of claim 14, wherein the population of encapsulated nanoparticles has a polydispersity in nanoparticle diameter of less than about 10%.

16. The method of claim 15, wherein the population of encapsulated nanoparticle polydispersity in nanoparticle diameter of less than about 5%.

17. The method of claim 16, wherein the population of encapsulated nanoparticles has a polydispersity in nanoparticle diameter of less than about 2%.

18. The method of claim 1, wherein the average diameter of the encapsulated nanoparticles is less than 50 nm.

19. The method of claim 18, wherein the average diameter of the encapsulated nanoparticles is less than 40 nm.

20. The method of claim 19, wherein the target size range is between about 20 nm and about 40 nm.

21. A method of transdermal drug delivery, comprising the steps of:
encapsulating a first agent in liposome vesicles to form a first population of encapsulated nanoparticles having a first average diameter;
encapsulating a second agent in liposome vesicles to form a second population of encapsulated nanoparticles having a second average diameter different from the first average diameter; and
topically administering the first and second populations of encapsulated nanoparticles for transdermal delivery of the first and second agents to a patient.

22. The method of claim 21, wherein the first agent is an anesthetic, an analgesic, an antibiotic, or a hormone.

23. The method of claim 22, wherein the second agent is an anesthetic, an analgesic, an antibiotic, or a hormone different from the first agent.

24. A pharmaceutical composition comprising:
a population of liposome encapsulated nanoparticles having an average diameter within a target size range capable of passive transport across a stratum corneum of a subject for transdermal delivery of the nanoparticles; and
a pharmaceutically acceptable carrier.

25. The pharmaceutical composition of claim 24, wherein the population of encapsulated nanoparticles has a polydispersity in nanoparticle diameter of less than about 10%.

26. The pharmaceutical composition of claim 24, wherein the population of encapsulated nanoparticles has a polydispersity in nanoparticle diameter of less than about 5%.

27. The pharmaceutical composition of claim 24, wherein the population of encapsulated nanoparticles has a polydispersity in nanoparticle diameter of less than about 2%.

28. The pharmaceutical composition of claim 24, wherein the average diameter of the encapsulated nanoparticles is less than 50 nm.

29. The pharmaceutical composition of claim 28, wherein the target size range is between about 20 nm and about 40 nm.

30. The pharmaceutical composition of claim 24, wherein the population of encapsulated nanoparticles comprises an anesthetic, an analgesic, an antibiotic, or a hormone.

31. The pharmaceutical composition of claim 24, wherein the population of encapsulated nanoparticles is a first population having a first average diameter within a first target size range, further comprising:
a second population of liposome encapsulated nanoparticles having a second average diameter within a second target size range.

32. The pharmaceutical composition of claim 31, wherein the second target size range is capable of passive transport across the stratum corneum of the subject for transdermal delivery of the nanoparticles.

33. The pharmaceutical composition of claim 32, wherein the first population of encapsulated nanoparticles has a first drug release profile, and the second population of encapsulated nanoparticles has a second drug release profile.

34. The pharmaceutical composition of claim 31, wherein the second population of encapsulated nanoparticles comprises a locally effective anesthetic, analgesic, antibiotic or hormone.
